(19) United States
(12) Patent Application Publication
(10) Pub. No.: US 2024/0425843 A1
(43) Pub. Date: Dec. 26, 2024

(54) MESOPOROUS SILICA BINDING PEPTIDES AND METHODS OF ENZYME IMMOBILIZATION USING THE SAME

(71) Applicant: ARCHER DANIELS MIDLAND COMPANY, Decatur, IL (US)

(72) Inventors: Padmesh Venkitasubramanian, Forsyth, IL (US); Candan Tamerler, Lawrence, KS (US); Kyle Boone, Lawrence, KS (US)

(21) Appl. No.: 18/339,183

(22) Filed: Jun. 21, 2023

(51) Int. Cl. C12N 11/06; C07K 7/08; C12N 9/20; C12N 9/90

(52) U.S. Cl. CPC C12N 11/06; C07K 7/08; C12N 9/20; C12N 9/90; C12Y 301/01003; C12Y 501/03; C07K 2319/20

(57) ABSTRACT

Described herein are peptides that bind to mesoporous silica substrates with high affinity. The peptides are useful for immobilizing proteins to such substrates by forming fusion proteins that include the silica binding peptides at the termini of the protein, exemplified herein with fusions to the C-terminus of several test proteins. The peptides may thus be used for making enzymatic reactor columns containing an immobilized enzyme over which a sample containing a substrate may be introduced as an input to the column and the reaction product obtained from the output of the column.

### INCORPORATION-BY-REFERENCE OF SEQUENCE LISTING

The contents of the text file named txt. INDLICKU07.US01.txt and the xml file named INDLICKU07.xml which were created on May 8, 2023 size, are hereby incorporated by reference in their entireties.

### TECHNICAL FIELD

The present invention relates to peptides that bind mesoporous silica substrates inclusive of mesoporous silica clays, which peptides are useful for immobilization of proteins onto mesoporous silica substrates by forming fusion proteins with the peptides.

### BACKGROUND OF THE INVENTION

Enzyme immobilization technologies have gained rapid development since the first commercial use of immobilized enzymes in the 1960s. Immobilized enzyme technology allows for continued reuse of the catalytic function of the enzyme in a reactor in the form of a column where the enzyme substrate is introduced at the input port of the column and the product of the enzymatic reaction emerges from the output port of the column. More generally, the ability to bind particular proteins on a substrate while not binding others allows for rapid purification of proteins from crude extracts.

Several techniques for enzyme immobilization have been deployed in the recent past. The most crucial part of the immobilization process is the selection of the appropriate immobilization method as it plays the biggest role in determining the enzyme activity and characteristics in a particular reaction. The major factors to consider for enzyme immobilization methods are avoidance of enzyme deactivation, the amount of binding, per weight of substrate, relative permanence of the binding, cost of the immobilization procedure, toxicity of immobilization reagents and the fluid flow properties of the substrate where such substrate is used in a column reactor. Exemplary enzymes that have been immobilized include lipases, peroxidases, isomerases and the like, which are typically immobilized on a porous matrix in a column so that the substrate for the enzyme can be introduced into the column as the input flow and the product of the enzymatic conversion flows out as the effluent flow.

Enzyme immobilization methods may be divided into two general classes namely, chemical and physical methods. Physical methods are characterized by weaker, non-covalent interactions such as hydrogen bonds, hydrophobic interactions, van der Waals forces, affinity binding, ionic binding or mechanical containment of enzyme within the matrix of the solid substrate. On the other hand, in chemical methods, involve formation of covalent bonds between the enzyme and support material typically achieved through ether thio-ether, amide or carbamate bonds directly between the enzyme and the substrate, or cross linking through a linker molecule bridging the substrate and enzyme. These two broad categories of immobilization technology can be divided into four techniques namely, adsorption and entrapment for the physical techniques, and covalent bonding and cross-linking for the chemical techniques. However, not one method is ideal for all molecules or purposes considering the inherently complex nature of protein structure. Moreover, the techniques mentioned above have some drawbacks. Adsorption techniques may result in gradual desorption of the enzyme from the substrate. Entrapment techniques require extremely small pores to entrap the enzyme, resulting in substrates with poor fluid flow. Chemical techniques may result in loss of enzyme activity, improper orientation on the substrate matrix, and use of expensive resins having suitable moieties for covalent cross linking.

Peptide tags provide a promising adsorption type solution that may overcome many of the problems with prior art methods. The use of peptides as tags for binding proteins to a substrate matrix has long been used as an important tool in molecular biology and diagnostic applications. Peptide tags allow the detection, purification and analysis of a particular protein or other binding partner that bind to certain proteins. In the context of molecular biology, peptide tags are made by forming a fusion protein containing the peptide tag typically at the N-terminus or C-terminus of the protein of interest. One commonly used peptide tag for this is polyhistidine, which strongly binds to nickel so that an enzyme or other protein may be quickly bound to a nickel containing substrate which allows rapid purification of the protein from a complex mixture. Nickel, alone is not a suitable substrate for immobilizing an enzyme for the purpose of conducting a catalytic reaction over a column because nickel is not a porous substrate through which a feedstock containing reactant can flow. Methods using nickel binding peptides therefore use porous polymeric resins covalently linked to nickel to overcome this problem, however, porous polymeric resins are costly.

The binding of peptide tags linked to an enzyme for use in a column reactor requires the binding partner to be a solid support material that is porous for effective fluid flow through the column. One of the most abundant and inexpensive substrates that could be used as column matrix material are silica containing substrates. Silica containing substrates include pure silica and clays that contain silica combined with various trace metals like aluminium, sulphur, calcium, magnesium, iron and thallium (collectively silicates). These substrates can be obtained in a variety of nonporous forms having different degrees of porosity. Microporous silicates, have an average pore size of less than 2 nm, mesoporous silicates have am average pore size in the range of 2 to 50 nm and macroporous silicates have an average pore size greater than 50 nm. There is limited prior art on the discovery of peptides that bind silicate substrates.

Naik, R. R., Brott, L. L., Clarson, S. J. & Stone, M. O. Silica-precipitating peptides isolated from a combinatorial phage display library. J. Nanosci. Nanotech. 2, 95-100 (2002) report the identification of several peptides capable of precipitating silica as elucidated from a phage display library.

U.S. Pat. No. 10,793,848 discloses an immobilized protein material comprising a protein that is immobilized on a glass material called controlled porosity glass (CPG) having a pore size of 10 to 300 nm. This disclosure teaches chemically reacting the CPG with a metal like nickel and binding a protein to the CPG using a polyhistidine peptide tag fused to the protein of interest. to the he disclosure also relates to the use of an immobilized enzyme material as a heterogeneous biocatalyst in chemical synthesis.

U.S. Pat. No. 10,882,885 discloses 35 peptides capable of precipitation of a protein onto a silica substrate when subject posttranslational modification. To function in this capacity, the peptides must be subjected to some post translational modification defined as any covalent modification to the R-group of an amino acid of the peptide, wherein the covalent modification generates a modified amino acid.

U.S. Pat. No. 9,404,097 discloses 7 peptides derived from naturally occurring protein sequences that can bind silica and are useful for polymerizing silica from a silica precursor in an aqueous solution. The peptide for synthesizing silica is said to form a self-assembled structure during silica synthesis, and thus can also be used to immobilize a protein onto a silica substrate.

US Pat. Pub. No. 20100158822 discloses 23 peptides having strong affinity for silica. The silica-binding peptides are stated to be be useful for preparation of peptide based-reagents suitable for delivery of a silica-coated particulate agents to a surface, such as body surface, for personal care and cosmetic applications.

There remains a need in the art identify new peptide sequences capable of binding silicate substrates and clays, particularly mesoporous silica substrates for use in immobilizing proteins onto silicate substrate.

### SUMMARY OF THE INVENTION

The present invention solves the forging problems by providing peptide sequences suitable for binding proteins, including enzymes, to a variety of mesoporous silica substrates, including mesoporous silica itself and several mesoporous silica clays. It is emphasized herein, that although some of mesoporous silica binding peptides may have been identified as extant in naturally occurring proteins, the present invention excludes such mesoporous silica binding peptides in the form they occur in naturally occurring proteins but instead focuses on use of mesoporous silica binding peptides as peptides to fuse to any other protein of interest for the purpose of binding the protein to mesoporous silica substrates.

In exemplary embodiments, the present invention provides a mesoporous silica binding peptide comprising an amino acid sequence selected from the group consisting of SEQ ID NOS; 1-16.

In exemplary embodiments, the mesoporous silica binding peptide is fused to the C-terminus of a protein. In certain exemplary embodiments, the mesoporous silica binding peptide further includes a linker peptide sequence fused between the mesoporous silica binding peptide and the protein. In certain particular embodiments the linker sequence is SGGGCGPXNGPC (SEQ ID NO: 17) where XN is a peptide sequence of a mesoporous silica binding peptide X having a length of N residues, wherein N is in the range of 8-18 residues.

In certain embodiments, the mesoporous silica binding peptide to which the peptide is binds consists essentially of silicon and oxygen, i.e., pure silica. In other embodiments the e mesoporous silica binding peptide to which the peptide binds is a clay that comprises on a molar basis, 20% to 35% silicon and 60% to 70% oxygen. In exemplary embodiments, the clay further includes on a molar basis, up to 10% of at least one trace metal selected from the group consisting of aluminium, calcium, gold, iron, magnesium, potassium, sulphur and thallium. In certain embodiments the clay further comprises up to 6% aluminium.

In another aspect, the invention provides for a protein fused to any of the forgoing mesoporous silica binding peptides. In typical embodiments, the mesoporous silica binding peptide is fused to a N-terminus or C-terminus of the protein. In exemplary embodiments the peptide is fused to the C-terminus of the protein. Certain embodiments may further include a linker peptide sequence fused between the mesoporous silica binding peptide and the protein.

In most desirable embodiments, the protein is an enzyme. In some exemplary embodiments the enzyme is a lipase. In other exemplary embodiments the enzyme uses a carbohydrate as substrate. In some exemplary embodiments the enzyme is a maltose binding protein. In another exemplary embodiment the enzymes is a tagatose epimerase.

Another aspect of the invention provides for a column comprising a mesoporous silica substrate bound to a protein fused to the inventive mesoporous silica binding peptides. In most desirable embodiments the protein is an enzyme. In many desirable embodiments the enzyme uses a carbohydrate as a substrate.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a table showing the chemical composition of example mesoporous silica substrates suitable for binding with peptides of the present invention as determined by scanning electron microscope electron diffraction spectroscopy (SEM/EDS). ND stands for “not detected”.

FIG. 2 is a table showing residual binding of some peptides of the present invention to some commercially available mesoporous silica substrates after incubating the substrates with the peptides and after washing (AW) the substrate to remove loosely bound peptides.

FIG. 3 shows the amino acid acid sequence of a triple fusion protein consisting of maltose binding protein (MBP, dark font) at the N-terminus fused to green fluorescence protein (GFPuv, light font) fused using a linker spacer SGGGCGP . . . GPC (italicized font) fused at the C terminus to the ADM111 peptide (SEQ ID NO:11) underlined Panel A, or to ADM18 peptide (SEQ ID NO: 8) underlined panel B.

FIG. 4 shows a fluorescence micrograph illustrating that maltose binding protein fused with green fluorescence protein alone MBP-GFP (Panel A) does not show retained binding to Tyrosil 300 after washing (lower panels) whereas a C-terminal fusion of the same fusion protein to the ADM111 peptide (SEQ ID NO:11) Pane B or to ADM18 peptide (SEQ ID NO: 8) Panel C, does retain binding to the Trysil 300 substrate after washing.

FIG. 5 is a chart showing the retention of binding to the mesoporous silica substrate Tryisil 300 of a protein fused to the mesoporous silica binding peptide according to SEQ ID NO;12 after extensive washing of the substrate to remove loosely bound material.

FIG. 6 is a chart showing the retention of binding to the mesoporous silica substrate Tryisil 300 of a protein fused to the mesoporous silica binding peptide according to SEQ ID NO;13 after extensive washing of the substrate to remove loosely bound material.

FIG. 7 is a chart showing the retention of binding to the mesoporous silica substrate Tryisil 300 of a protein fused to the mesoporous silica binding peptide e according to SEQ ID NO;14 after extensive washing of the substrate to remove loosely bound material.

FIG. 8 is a chart showing the retention of binding to the mesoporous silica substrate Tryisil 300 of a protein fused to the mesoporous silica binding peptide according to SEQ ID NO;15 after extensive washing of the substrate to remove loosely bound material.

FIG. 9 is a chart showing the retention of binding to the mesoporous silica substrate Tryisil 300 of a protein fused to the mesoporous silica binding peptide according to SEQ ID NO;16 after extensive washing of the substrate to remove loosely bound material.

FIG. 10 is a chart showing the retention of binding to the mesoporous silica substrate Oil Dri Perform 5000 of a protein fused to the mesoporous silica binding peptide according to SEQ ID NO;12 after extensive washing of the substrate to remove loosely bound material.

FIG. 11 is a chart showing the retention of binding to the mesoporous silica substrate Oil Dri Perform 5000 of a protein fused to the mesoporous silica binding peptide according to SEQ ID NO;13 after extensive washing of the substrate to remove loosely bound material.

FIG. 12 is a chart showing the retention of binding to the mesoporous silica substrate Oil Dri Performa 5000 of a protein fused to the mesoporous silica binding peptide according to SEQ ID NO; 14 after extensive washing of the substrate to remove loosely bound material.

FIG. 13 is a chart showing the retention of binding to the mesoporous silica substrate Oil Dri Performa 5000 of a protein fused to the mesoporous silica binding peptide according to SEQ ID NO; 15 after extensive washing of the substrate to remove loosely bound material.

FIG. 14 is a chart showing the retention of binding to the mesoporous silica substrate Oil Dri Perform 5000 of a protein fused to the mesoporous silica binding peptide according to SEQ ID NO;16 after extensive washing of the substrate to remove loosely bound material.

FIG. 15 shows the predicted tertiary structure of example protein tagatose epimerase having mesoporous silica binding peptide ADM111 fused at either the C terminus or N terminus of the protein indicating no substantial change in tertiary structure of the functional portion of the enzyme in either case.

### DETAILED DESCRIPTION OF THE INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

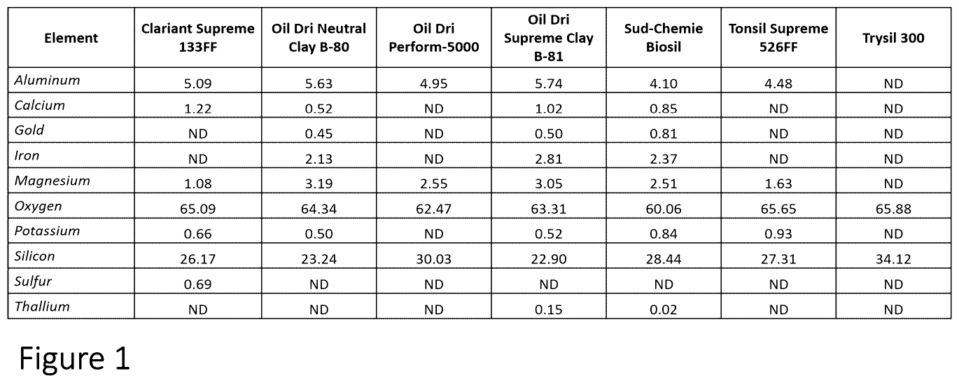

Disclosed herein are peptides that bind to mesoporous silica substrates, which includes silica and silica containing clays, with sufficient affinity for use as peptide tags for immobilizing protein onto such substrates. As used herein, a mesoporous silica substrate contains, on a molar basis, 20% to 35% silicon and 60% to 70% oxygen with or without additional mineral elements and has an average pore size of 2 nm to 50 nm by conventional definition or from 2 nm to 100 nm as may be used in practical embodiments. One non-limiting example of a commercially available pure silica mesoporous silicate substrate is Tysil 300™. Non-limiting types of other mineral elements in silicate clays include aluminium, calcium, gold, iron, magnesium, potassium, sulphur and thallium. Non-limiting examples of commercially available mesoporous silicate clays include Clariant Supreme 133FF™, Oil Dri Neutral Clay B-80™, Oil Dri Perform 5000™, Oil Dri Supreme Clay B-81™, Sud Chemie Biosil™, and Tonsil Supreme 526FF™. These clays may be grouped into two categories—low silicate clays having 25% or less of silicon content and high silica clays having greater than 25% silicon. FIG. 1 is a table showing the chemical composition of these example materials as determined by scanning electron microscope electron diffraction spectroscopy (SEM/EDS).

The peptides of the present disclosure were initially conceived using a computer model based on CLN-MLEM2, which is a rough set theory method for inducting rules that are relevant to the occurrence of classification labels in tabular data, which is used for analysis of patterns of dipeptides and tripeptides classified as exhibiting low, medium or high binding ((Boone et al, 2018). In addition to building relevant rules, the features selected for the rules have maximum relevance for distinguishing between the different classification labels from other available features in a data table. The features selected by the CLN-MLEM2 method are used in building multiple linear regression models describing the adsorption fraction as a function of alignment score and key physicochemical properties that are built in R using a MASS library and the DAAG library for cross-fold validation. The linear models are stepwise reduced from the collection of features from CN-MLEM2 and the alignment score such that features are removed from the linear model until the performance of the model sufficiently degraded. Sequence data for the silica precipitating peptides identified by Naik, R. R., Brott, L. L., Clarson, S. J. & Stone, M. O. J. Nanosci. Nanotech. 2, 95-100 (2002) was loaded into this model.

Peptide sequences predicted by the model to be capable of binding silicate substrates were synthesized to test binding by using an Aapptec Focus XC peptide synthesizer (Aapptec). The peptide-resins are assembled on Wang resins with C-terminal amino acids using Fmoc chemistry. The N-terminal Fmoc deprotection is performed by treatment with 20% piperidine/dimethylformamide (DMF) in a 0.2 mmol reaction scale with mixing and nitrogen gas bubbling. Effective removal of the Fmoc protecting group is monitored by UV spectroscopy. The peptide-resin is filtered, and the 20% piperidine/DMF solution is added repeatedly until complete deprotection is achieved as quantified by UV spectroscopy. Typically, two cycles of deprotection were found to be sufficient. The peptide-resins are then washed with DMF. Activation of 0.2 M amino acids/DMF is performed by addition of 0.2M 2-(1H-benzotriazol-1-yl)-1,1,3,3-tetramethyluronium hexafluorophosphate (HBTU)/DMF (2 equiv.) in a measuring vessel then added to the reaction vessel containing the deprotected peptide-resin for a 45-minute coupling reaction. The coupling step is completed twice to ensure addition of the desired amino acid. The procedure is repeated until the complete peptide is assembled on the solid resin support.

Following synthesis, the peptide-resin is removed from the reaction vessel using DMF. DMF is removed from the peptide-resin by washing with ethanol and drying on a course grained glass fritted Buchner funnel. The dried resin is transferred to a glass volumetric flask followed by addition of a cleavage cocktail (15 mL/1 gram of resin) for two hours with gentle stirring to remove the peptide from the solid support and remove the side chain protecting groups. The standard cleavage cocktail is trifluoroacetic acid (TFA)/triisopropylsilane (TIS)/water (95:2.5:2.5, % vol/vol/vol). In order to remove side chain protecting groups from peptides containing hystine or cysteine 2.5% thioanisole and 2.5% 1,2-ethanedithiol are added to the cocktail and for peptides containing methionine, tyrosine, or arginine 5% phenol is added. The cleavage products are filtered in a glass Buchner funnel and crude peptide product is isolated by precipitation in cold ether (30 mL). The crude peptide is pelleted by centrifugation (2000 rpm for 2 minutes), the supernatant is removed, the pellet is re-suspended in ether and recentrifuged for a total of four times. Following ether washes the crude peptide products are lyophilized. Following the peptide synthesis, support material characterization is conducted. Surface area, pore size distribution and pore volume are conducted with nitrogen gas through BET multipoint analysis using a Quantichrome Nova 2200e. Solid state NMR analysis is conducted on the support material to determine the connectivity of the silicate units. The ssNMR study is carried out with a 400 MHz Bruker AVIII spectrometer, equipped with a 4 mm two-channel MAS probe. The 4 mm zircon rotor is sealed with a KelF drive cap (Wilmad Lab-glass, Vineland, NJ). Calibration of MAS is done with KBr. All spinning speeds are 4 kHz.

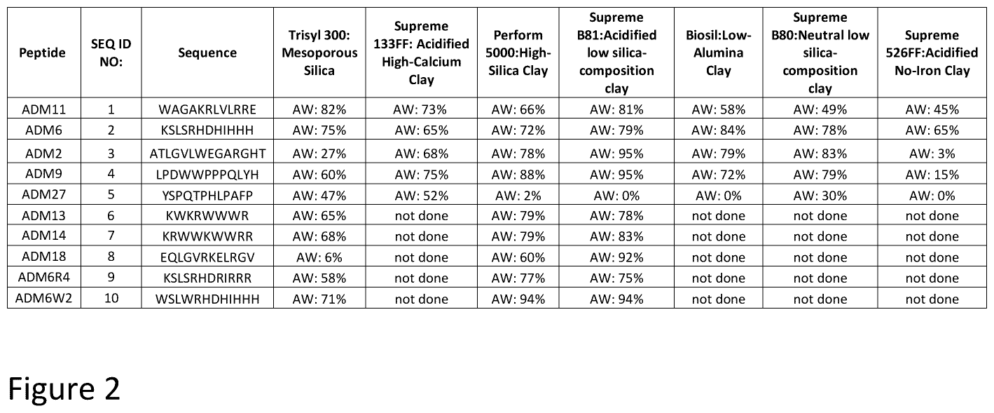

Generation 1 CLN-MLEM2 devised peptides. Initially, 10 candidate peptides (SEQ ID NOs: 1-10) were identified as remaining bound to at least one mesoporous silica substrate at a level of at least 50% after being subject to substrate adsorption experiments that were conducted as follows. 200 μL of each peptide solution with a peptide concentration of 840 μM and 200 μl of the silica clay material stock containing 0.25 mg of substrate was added to 1.5 mL microtube. Samples without support material (200 μl peptide solution and 200 μL water) and samples without peptide (200 μl of the support material stock and 200 μl water) were prepared to determine the recovery of the peptide from the microtube with no support material and the false positive concentration from the support, respectively. Incubation was carried out on a plate shaker on a 2.5 Hz setting for 16 hours. Microtubes were centrifuged at 1500 g for 1 hour to sediment the support material particles. Supernatant from each tube was transferred to a 600 μl microtube. The amount of peptide present in each sample was recorded in 4 replicates by measuring the average absorbance value taken at 220 nm (or 280 nm when applicable). The amount of peptide present was calculated through calibration curves of diluted peptide stock samples. The initial substrate adsorption fraction was calculated as the measurement difference between the initial nanomoles added from the peptide stock solution minus the nanomoles recovered after centrifugation minus the nanomoles determined to have been adsorbed to the tube from the blank measured. The adsorption fraction was calculated as the molar percentage of peptide adsorbed. To determine the strength of binding to the substrate, the substrate pellet recovered from the centrifugation step was washed by resuspension in 200 μl of water and the forgoing procedure was repeated to determine the amount of peptide remaining on the substrate after washing (AW). The percentage of peptides that remained bound to a panel of different mesoporous silica substrates after washing is summarized in the table shown in FIG. 2 for SEQ ID NOs: 1-10. Although the binding to various substrates varied, each of SEQ ID NO:s 1-10 retained at least 50% binding to at least one substrate after washing.

Specially devised peptide and spacer. Based on the results of the generation 1 peptides output by the CLN-MLEM2 system and knowledge of constraints on extracellular disulfide bond formation, the inventors devised and synthesized a peptide designated ADM111 (WALRRSIRRQSY, SEQ ID NO: 11) that would have increased flexibility when used with constrained disulfide bond linker types that should be more stable in extracellular environments. An example of such a constrained disulfide linker is the linker spacer designated herein as “C Linker”, which has the sequence SGGGCGPXGPC (SEQ ID NO: 17) where X is a place holder for the mesoporous silica binding peptide (e.g., ADM111) having a length of 8-18 residues.

Fusion Proteins. To determine whether the mesoporous silica binding peptides of the present invention would function to immobilize proteins onto mesoporous silica substrates, several fusion proteins were made that contained selected silica binding peptides linked to the C terminus of various test proteins. One such test protein is itself a fusion protein consisting of a maltose binding protein (MBP) fused at its C terminus to green fluorescence protein (GFPuv) having the sequence according to SEQ ID NO: 18). The MBP-GFPuv fusion protein has a residue length that is exemplary of many industrially important enzymes and retains the MBP ability to bind amylose when fused to a protein at its C terminus making it easy to purify over an amylose column, while GFPuv retains fluorescence activity when fused to another protein at its N terminus making it easy to detect binding to a substrate by detecting fluorescence after incubating the substrate with the fusion protein and washing the substrate to remove unbound material.

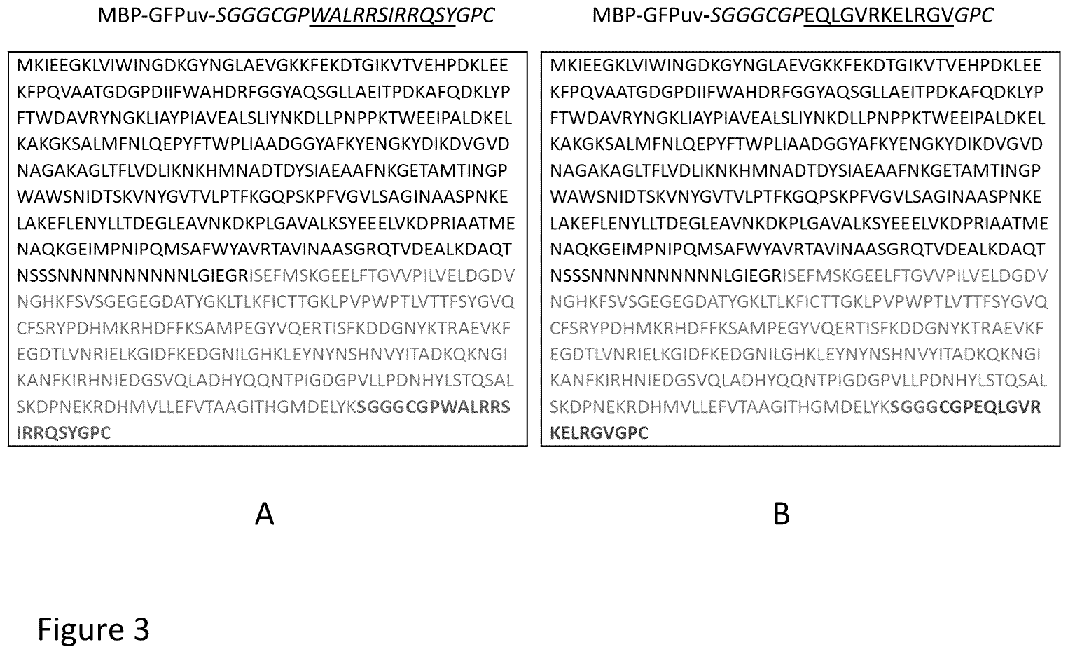

DNA sequences were synthesized that encode the MBP-GFPpuv protein alone or the same fused at the C terminus to ADM111 (SEQ ID NO: 11) or ADM18 (SEQ ID NO: 8) through the C-Linker spacer, expressed and tested as described in Yuca et al. Biotechnology & Bioengineering, 108 (5) 1021-103, (2011). The protein sequence of these constructs is shown in FIG. 3. Clarified cell lysates containing the expressed MBP fusion proteins in 20 mM sodium phosphate buffer (pH 7.2) and 200 mM NaCl were loaded onto an amylose column equilibrated with the same buffer and eluted and eluted with the same buffer but containing 0.5 M NaCl and maltose. The maltose was removed by ultrafiltration and the retentate and filter were washed with the 20 mM sodium phosphate buffer (pH 7.2) and 200 mM NaCl to recover the fusion protein.

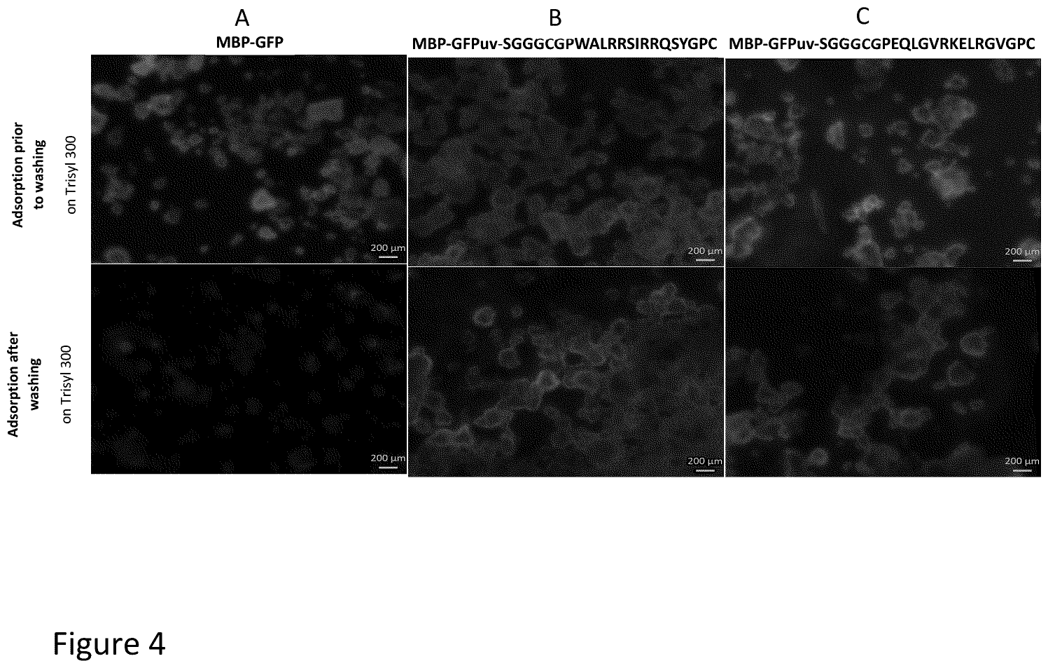

The purified fusion proteins were assayed for the ability to bind Perform 5000, Supreme B81 and Trysil 300, silica substrates before and after washing as previously described for the peptide sequences alone. The amount of fluorescence emitted from the substrate was measured using a fluorescence detector from Fisher Scientific. With Perform 5000, 91% of the fluorescence was retained after the washing for both the ADM111 and ADM18 peptide fusions. With Supreme B81, 90% was retained with ADM111 and 84% was retained with ADM18. With Trysil 300, 90% was retained with ADM111 and 87% was retained with ADM18. FIG. 4 shows fluorescent micrographs indicating that fusion proteins with both ADM111 and ADM18 were retained on Trysil 300 after washing while the MBP-FFPuv protein lacking the fusion peptide was not.

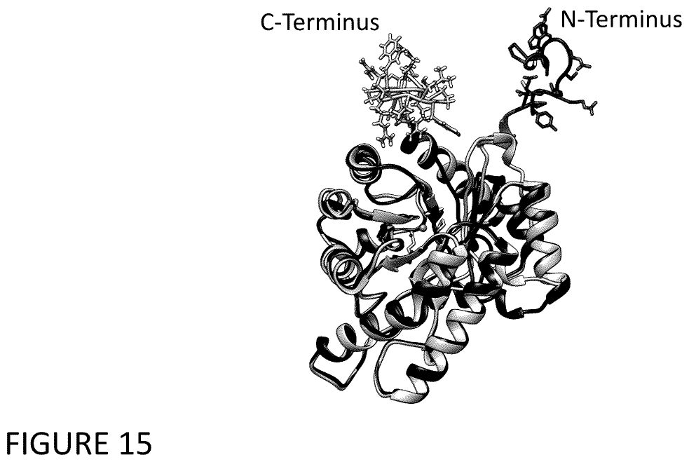

Another test fusion protein was a tagatose epimerase having the amino acid sequence according to SEQ ID NO: 19. Tagatose epimerase (a.k.a allulose or fructose epimerase) is used in production of the rare sugar allulose by epimerization of fructose in an aqueous reaction environment. A fusion protein that included tagatose epimerase was made by synthesizing a DNA sequence that include ADM111 fused to the C-terminus of tagatose epimerase with the C Linker spacer that was expressed in E. coli BL21 (DE3) using an expression vector under a T7 promoter. After growth and induction of expression with IPTG (1 mM) a crude extract of the cells containing vector expression the tagatose epimerase protein was made. The crude extract was mixed with Trysil 300 and the enzymatic activity of tagatose epimerase was measured before and after washing the substrate by adding fructose to the substrate and detecting the amount allulose synthesized. The washed substrate retained 80% of the enzymatic activity detected with the unwashed substrate indicating at least 80% of the tagatose epimerase remained bound to the substrate after washing. Another fusion protein was made with ADM111 fused to the N-terminus of tagatose epimerase, which would also bind to Tyrsyil 30 and retain activity because fusion of the peptide at either the N or C terminal of tagatose epimerase is not predicted to substantially alter the structure of the enzyme as shown in FIG. 15.

Yet another test protein tests a lipase from Thermoascus aurantiacus (TAL) having the aminos acid sequence according to SEQ ID NO: 20. This lipase is used in industrial scale production of fatty acids and interesterified vegetable oils in a semi aqueous/semi hydrophobic reaction environment. A DNA sequence was synthesized that encodes TAL with ADM18 was fused to the C-terminus of TAL through a small intervening linker sequence SGGG. The DNA construct was introduced into an inducible expression vector for expressing the TAL protein fusion in A. oryzae. The cells were grown, induced for expression and crude extract was obtained. The crude extract was mixed with Trysil 300 and the silica substrate was washed once, twice, three times or 4 times. After washing, the Trysil 300 substrate was incubated with the substrate of the lipase. Even after the 4th wash at least 90% of the initially bound lipase activity remained detectable indicating that ADM18 was able to immobilize the lipase onto Tyrisil 300.

The Immobilized enzyme was used to evaluate interesterification of refined bleached soy oil and fully hydrogenated soybean oil (73:27) using the method published in AOAC method Cd16b093. The solid fat content in the reaction mixture was measured and reported as INU/g. The immobilized enzyme activity was compared to two commercially available immobilized lipase. As seen from the table below the immobilized TAL lipase fused with the ADM18 had higher activity compared to the commercially available immobilized lipases

| Sample | IUN/g |
|---|---|
| Commercial Enzyme 1 rep 1 | 21 |
| Commercial Enzyme 1 rep 2 | 27 |
| Immobilized TAL lipase: ADM18 rep 1 | 565 |
| Immobilized TAL lipase: ADM18 rep 2 | 555 |
| Commercial enzyme 2 rep 1 | 376 |
| Commercial enzyme 2 rep 2 | 398 |

Generation 2 CLN-MLEM2 devised peptides. Substrate binding capacity of all the samples was entered into the CLN-MLEM2 model to generate sequences for further candidate peptides likely to bind the substrates. Several were subjected to the same initial screening for silicate binding as the generation 1 peptides using Trisyl-300 silica, and Oil Dri Performa-5000 silica clay as substrates. In addition, several candidate peptides were subjected to the following more rigorous binding and wash protocol. 500 μl of a 500 UM peptide solution in 15 mM Tris-HCl buffer pH 7.4 was incubated with 50 mg of mesoporous silica substrate material. Each sample was incubated at STP for 16 hours. The samples were centrifuged and the supernatant was removed. The remaining pellet was subjected to sequential washes with 500 μl 15 mM buffered solution at varying pHs (4.5/7.4/8.5. The sash step were repeated 7 times at room temperature and on wash 8 the temperature of the wash was heated to 50° C. This step was repeated for wash 9. Wash 10 was heated to 70° C. The final wash (no. 11) was heated to 90° C. The amount of peptide recovered in each wash was determined by HPLC chromatography over as peptide specific reverse phase C-18 resin. The sum of the amounts recovered in each was subtracted from the amount originally loaded onto the substrate to calculate the amount retained by the substrate.

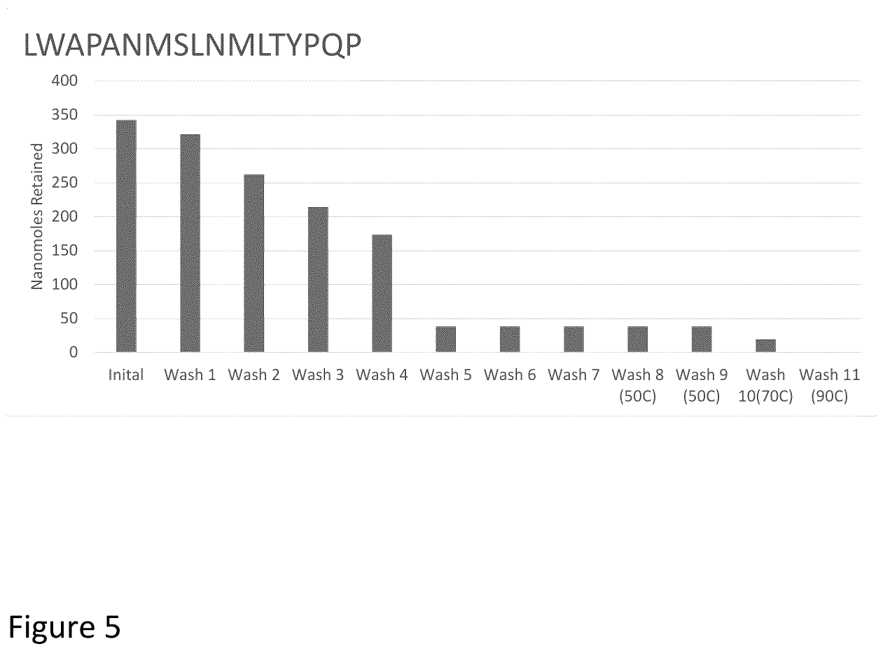

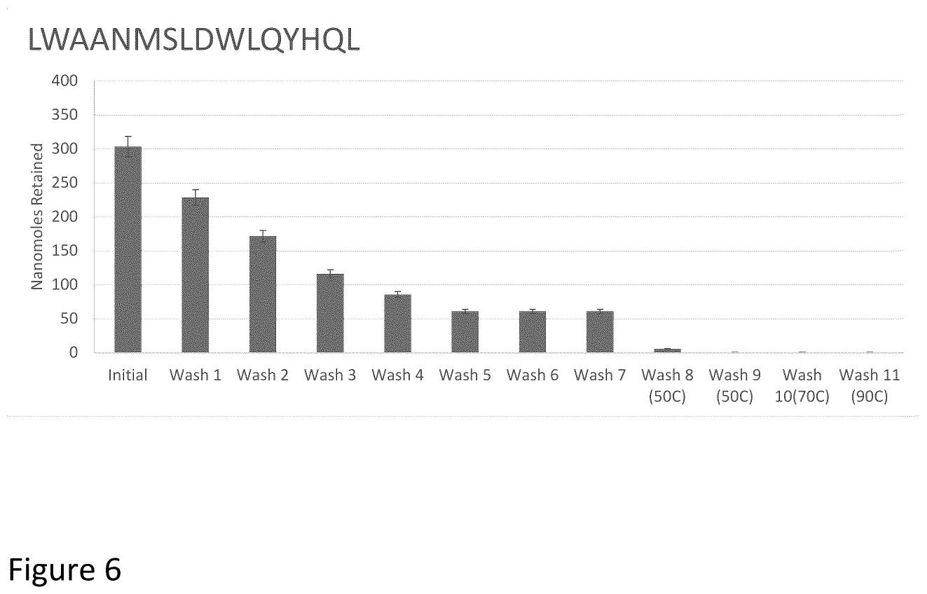

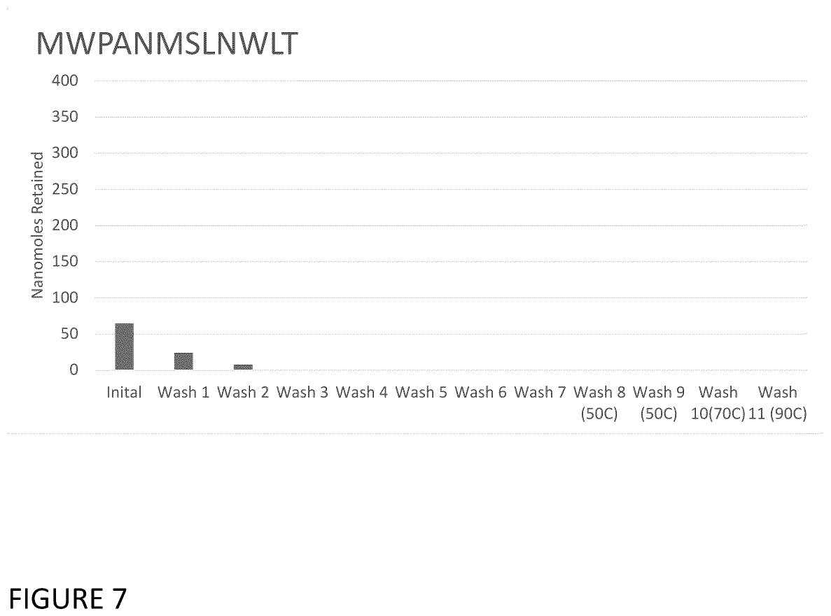

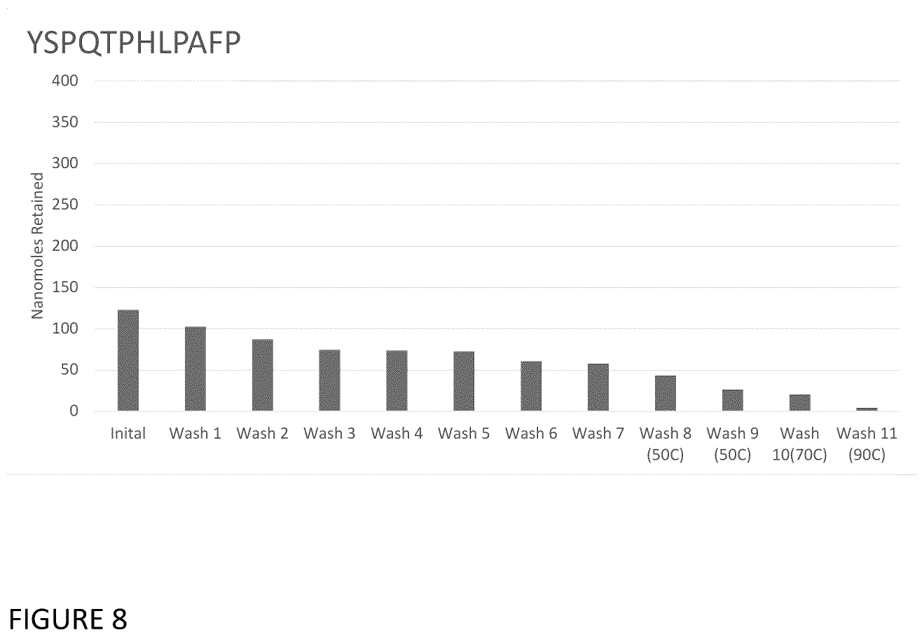

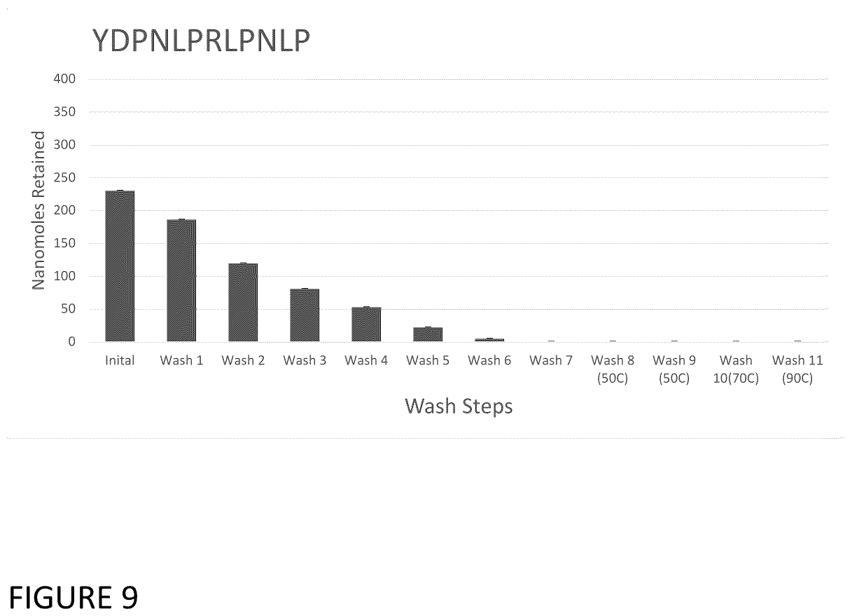

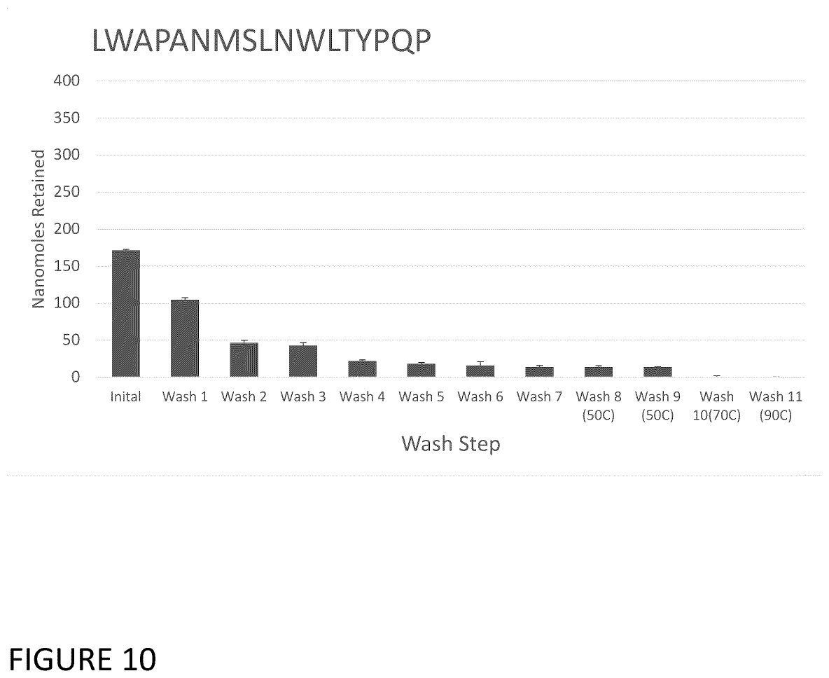

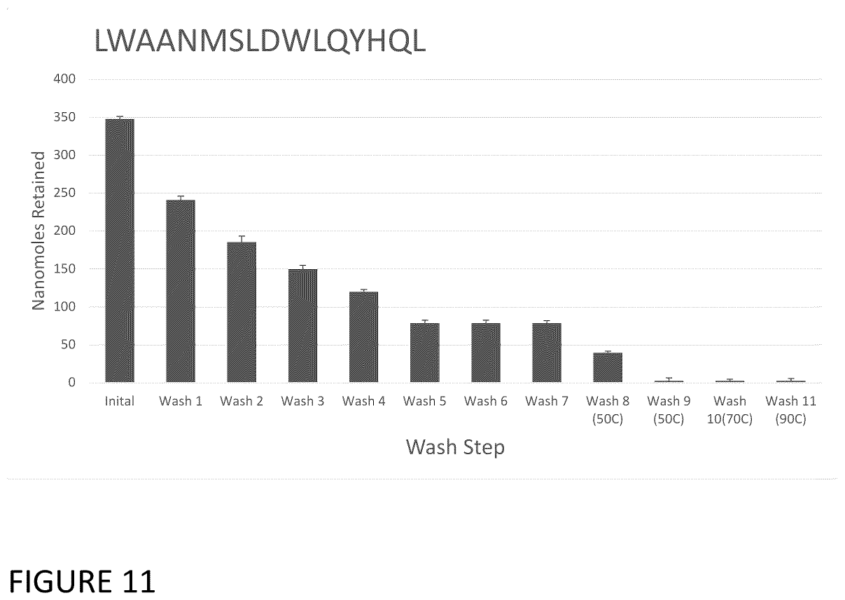

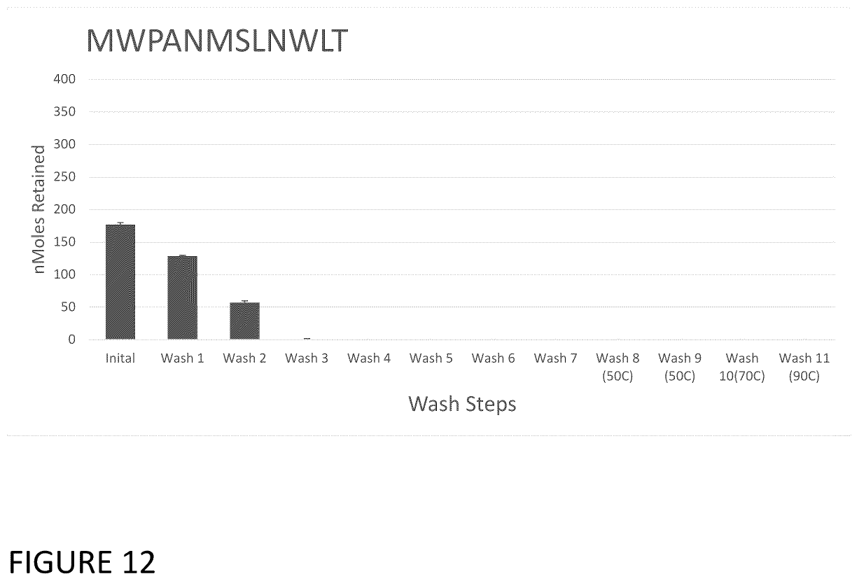

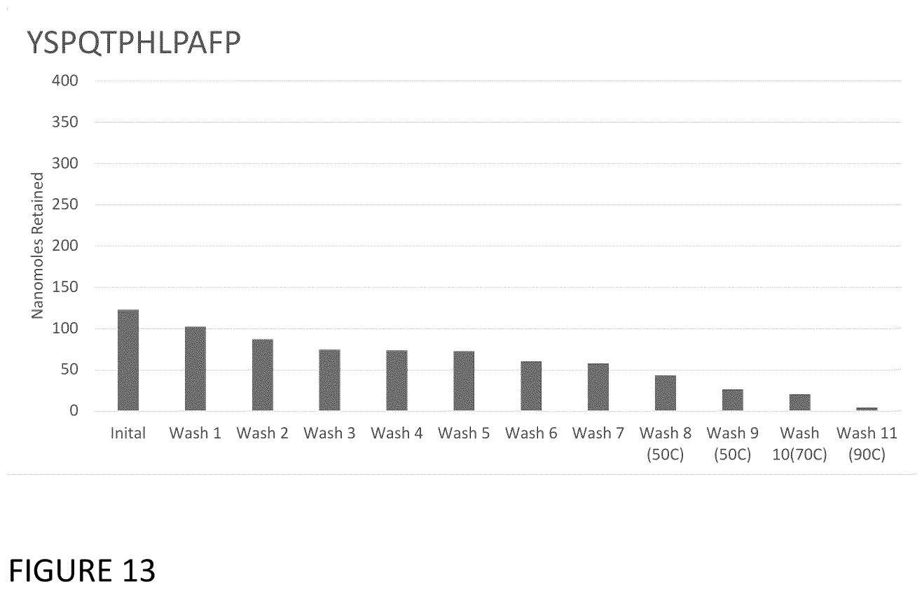

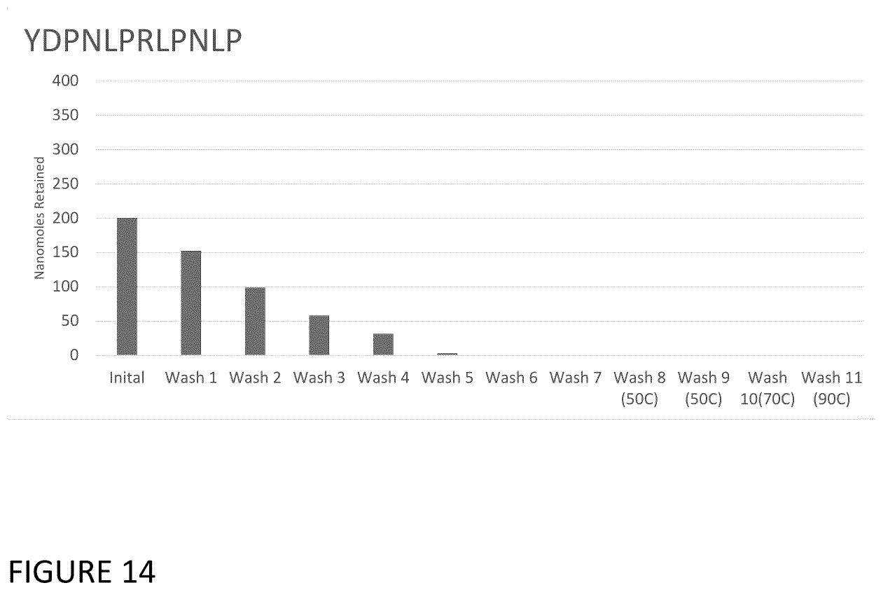

FIGS. 5 through 14 shows that for each of LWAPANMSLNWLTYPQP (SEQ ID NO: 12, LWAANMSLDWLQYHQL (SEQ ID NO: 13), YDPNLPRLPNLP (SEQ ID NO: 14), MWPANMSLNWLT (SEQ ID NO:15), and YSPQTPHLPAFP (SEQ ID NO: 16) at least 50% of the initial peptide load was retained after a first wash. Of these, SEQ ID NO: 12, 13 and 16 demonstrated a persistent binding Trysil-300 even after eleven washing steps while SEQ ID NO: 12 and 13 also showed such persistent to binding to Oil Dri Perform-500, indicating in each case that a thin film of these peptides can remain tenaciously bound to these substrates.

While only certain features of the invention have been illustrated and described herein, many modifications and changes may occur to those skilled in the art. It is understood that the appended claims are intended to cover all such modifications and changes as falling within the scope of the present invention.

### REFERENCES

Boone, K., Camarda, K., Spencer, P. and Tamerler, C., 2018. Antimicrobial peptide similarity and classification through rough set theory using physicochemical boundaries. BMC bioinformatics, 19 (1): 1-10.
Naik, R. R., Brott, L. L., Clarson, S. J. & Stone, M. O. Silica-precipitating peptides isolated from a combinatorial phage display library. J. Nanosci. Nanotech. 2, 95-100 (2002).
Sarikaya M, Tamerler C, Jen AKY, Schulten K, Baneyx F. Molecular biomimetics: nanotechnology through biology. Nature materials. 2003; 2 (9): 577-85
Smith, G. P. Filamentous fusion phage: Novel expression vectors that display cloned antigens on the virion surface. Science 228, 1315-1317 (1985).
Whaley, S. R., English, D. S., Hu, E. L., Barbara, P. F. & Belcher, M. A. Selection of peptides with semiconducting binding specificity for directed nanocrystal assembly. Nature 405, 665-668 (2000).

1. A mesoporous silica binding peptide comprising an amino acid sequence selected from the group consisting of SEQ ID NOS: 1-16, excluding such peptides as linked in a naturally occurring protein sequence.

2. The mesoporous silica binding peptide of claim 1, wherein the mesoporous silica binding peptide is fused to a termini of a heterologous protein.

3. The mesoporous silica binding peptide of claim 2, wherein the mesoporous silica binding peptide is fused to the C-terminus of the heterologous protein.

4. The mesoporous silica binding peptide of claim 2, further comprising a linker peptide sequence fused at least partially between the mesoporous silica binding peptide and the protein.

5. The mesoporous silica binding peptide of claim 4 wherein the linker peptide sequence is SGGGCGPXGPC (SEQ ID NO: 17) where X is a place holder for a peptide sequence of 8 to 18 residues

6. The mesoporous silica binding peptide claim 5 wherein the mesoporous silica binding peptide is fused to the C-terminus of the heterologous protein.

7. The mesoporous silica binding peptide of claim 1 wherein the mesoporous silica to which the peptide is binds consists essentially of silicon and oxygen.

8. The mesoporous silica binding peptide of claim 1 wherein the mesoporous silica to which the peptide binds is a clay that comprises on a molar basis, 20% to 35% silicon and 60% to 70% oxygen.

9. The mesoporous silica binding peptide of claim 8 wherein the clay further includes on a molar basis, up to 10% of at least one trace metal selected from the group consisting of aluminium, calcium, gold, iron, magnesium, potassium, sulphur and thallium.

10. The mesoporous silica binding peptide of claim 8 wherein the clay further comprises up to 6% aluminium.

11. The mesoporous silica binding peptide of claim 1 wherein the peptide has an amino acid sequence selected from the group consisting of SEQ ID NOs: 1-16.

12. The mesoporous silica binding peptide of claim 1 wherein the peptide has an amino acid sequence selected from the group consisting of SEQ ID NOs: 12, 13 and 16.

13. A protein fused to the mesoporous silica binding peptide of any of claims 1 to 11 claim 1.

14. The protein according to claim 13 wherein the mesoporous silica binding peptide is fused to the C-terminus of the protein.

15. The protein according to claim 13 further comprising a linker peptide sequence fused between the mesoporous silica binding peptide and the protein.

16. The protein according to claim 15 wherein the linker peptide sequence is SGGGCGPXGPC (SEQ ID NO: 17) where X is a place holder for a peptide sequence of 8 to 18 residues.

17. The protein according to claim 16 wherein the mesoporous silica binding peptide is fused to the C-terminus of the protein.

18. The protein of claim 13 wherein the wherein the peptide has an amino acid sequence selected from the group consisting of SEQ ID NOs: 1-16.

19. The protein of claim 13 wherein the wherein the peptide has an amino acid sequence selected from the group consisting of SEQ ID NOs: 12, 13 and 16.

20. The protein according to claim 13 wherein the protein is an enzyme.

21. The protein according to claim 13 wherein the enzyme is a lipase.

22. The protein according to claim 13 wherein the enzyme is a tagatose epimerase.

23. A column comprising a mesoporous silica substrate bound to a protein fused to the mesoporous silica binding peptide of claim 1.


SEQUENCE LISTING (20 sequences)

SEQ ID NO: 1 - protein, 12 aa, synthetic construct
WAGAKRLVLRRE

SEQ ID NO: 2 - protein, 12 aa, synthetic construct
KSLSRHDHIHHH

SEQ ID NO: 3 - protein, 14 aa, synthetic construct
ATLGVLWEGARGHT

SEQ ID NO: 4 - protein, 12 aa, synthetic construct
LPDWWPPPQLYH

SEQ ID NO: 5 - protein, 12 aa, synthetic construct
YSPQTPHLPAFP

SEQ ID NO: 6 - protein, 8 aa, synthetic construct
KWKRWWWR

SEQ ID NO: 7 - protein, 9 aa, synthetic construct
KRWWKWWRR

SEQ ID NO: 8 - protein, 12 aa, synthetic construct
EQLGVRKELRGV

SEQ ID NO: 9 - protein, 12 aa, synthetic construct
KSLSRHDRIRRR

SEQ ID NO: 10 - protein, 12 aa, synthetic construct
WSLWRHDHIHHH

SEQ ID NO: 11 - protein, 12 aa, synthetic construct
WALRRSIRRQSY

SEQ ID NO: 12 - protein, 17 aa, synthetic construct
LWAPANMSLNWLTYPQP

SEQ ID NO: 13 - protein, 16 aa, synthetic construct
LWAANMSLDWLQYHQL

SEQ ID NO: 14 - protein, 12 aa, synthetic construct
YDPNLPRLPNLP

SEQ ID NO: 15 - protein, 12 aa, synthetic construct
MWPANMSLNWLT

SEQ ID NO: 16 - protein, 12 aa, synthetic construct
YSPQTPHLPAFP

SEQ ID NO: 17 - protein, 11 aa, synthetic construct
  VARIANT 8: note = X can be any amino acid
SGGGCGPXGPC

SEQ ID NO: 18 - protein, 628 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRISEFMSKGEELFTGWPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 19 - protein, 291 aa, synthetic construct
MNKVGMFYTYWSTEWMVDFPATAKRIAGLGFDLMEISLGEFHNLSDAKKRELKAVADDLGLTVMCCIGLKSEYDFASPDKSVRDAGTEYVKRLLDDCHLLGAPVFAGLTFCAWPQSPPLDMKDKRPYVDRAIESVRRVIKVAEDYGIIYALEVVNRFEQWLCNDAKEAIAFADAVDSPACKVQLDTFHMNIEETSFRDAILACKGKMGHFHLGEANRLPPGEGRLPWDEIFGALKEIGYDGTIVMEPFMRKGGSVSRAVGVWRDMSNGATDEEMDERARRSLQFVRDKLAS

SEQ ID NO: 20 - protein, 297 aa, synthetic construct
MFNSVAKWAVTALTLGLFVSAAPYKPERRDVSAELLQQFNLFEQYAAAAYCEENFNSTGTKLTCSAGNCPLVEAADTKTIDEFTDSSYGNVTGFLAVDNTNKLIVLSFRGTRSIDTWISNLDFTLKDIGDICSGCEIHNGFWKSWNSVSDDLTSRLQSTVSEYPDYAIIFTGHSAGAAVATVGTLVLRKVGYAVDLYPYGCPRIGNGQLAEYMTTQTPGTNYRITHTDDIVPRLPPEWAGYSHYSPEYWITSPNNVTVTTADIQVIQGIDSDAGNAGTDGLSTDAHGWYFGPISACQ